(19) United States
(12) Patent Application Publication
(10) Pub. No.: US 2004/0069707 A1
(43) Pub. Date: Apr. 15, 2004

(54) Method of isolating a charged compound

(76) Inventor: Michael Naldrett, Norwich (GB)

Correspondence Address: SUGHRUE MION, PLLC, 2100 PENNSYLVANIA AVENUE, N.W., SUITE 800, WASHINGTON, DC, 20037, US

(21) Appl. No.: 10/468,799

(22) PCT Filed: Feb. 21, 2002
(86) PCT No.: PCT/GB02/00770

(30) Foreign Application Priority Data
Feb. 22, 2001 (GB) 01004429.6

(51) Int. Cl. B01D 15/00; B01D 21/26; B01D 61/00

(52) U.S. Cl. 210/638; 210/660; 210/781

(57) ABSTRACT

A method of isolating a charged, especially proteinaceous, compound comprises providing a prepared sample of the compound in solution, placing the prepared sample in contact with a membrane so limited in effective adsorptive surface area as to enable reversible binding to the membrane of the charged compound in the sample presented to the adsorptive surface such that solvent is removable therefrom and the charged compound is left substantially reversibly bound thereto, and eluting the compound from the membrane. Eluted compounds can then be directly subjected to mass spectrometric analysis for charcterisation of, for example, individual peptides.

The present invention relates to a method of isolating a charged compound, especially a proteinaceous compound such as a peptide, polypeptide or protein.

Various methods of isolating charged, for example proteinaceous, compounds are known, including the recovery of proteins from prepared samples by use of membranes constructed to extract target substances by reverse phase or ion exchange chromatography. In the latter case, the ion exchange membrane typically consists of a discoid porous matrix of modified cellulose with a covalently attached, positively or negatively charged functional group, for example sulphonic acid or quaternary ammonium, imparting a cationic or anionic character to the membrane. The membrane is placed or retained in the base of a receptacle or column for reception of a sample in liquid phase to be driven through the membrane under the effect of, for example, centrifugal force. The receptacle itself an be removably located in a centrifuge tube enclosing a collecting chamber for collection of media passed through the membrane. In the field of protein recovery, such a membrane system is commonly used for extraction of proteins from specific tissues or other organic materials, purification or concentration of isolated proteins and polypeptides and removal of non-proteinaceous elements such as endotoxins, sugar, salts and the like. For these and other such purposes, equilibration of a membrane containing a functional group appropriate to ion exchange with the target proteinaceous compound is initially carried out by driving a buffer solution of a preselected appropriate kind and pH value through the membrane to bring the pH level thereof to that of a protein-containing sample and subsequently a further volume of the solution, but now containing the sample, is driven through the equilibrated membrane to trigger ionic binding of the protein constituent of the sample to the matrix by anion or cation (depending on the pH value and isoelectric point of the protein and selected membrane functional group) interaction. The membrane is then washed by a further volume of the buffer solution and finally the bound protein molecules are recovered by elution with the same buffer containing a high salt concentration or with a buffer solution, or series of buffer solutions, of ascending or descending pH, thus enabling subsequent recovery of the protein. The washing and elution steps are both performed in similar manner to the preceding steps, such as by centrifuging the column and tube assembly. The afore-mentioned procedure using ion exchange membranes has proved very effective in processing protein-loaded samples for the purposes mentioned. However, the membranes hitherto have not been considered practicable for concentrating very small amounts of charged compounds, such as proteins, from large volumes of dilute solutions. Use of known membranes in connection with analysis of proteins in dilute samples is, to all intents and purposes, impossible when the requirement is for concentration of analysable quantities of proteins in the samples into small volumes, such as from about 0.5 μl to about 5.0 μl volumes. Furthermore, the initial equilibration and intermediate washing steps associated with the known methods extend the overall recovery time, which can be particularly disadvantageous in proteomics procedures where high numbers of samples need to be rendered suitable for sensitive analysis, for example by mass spectrometric techniques.

For that purpose, widespread use has been made of, for example, pipettes with removable tips having, at the free end, a micro-volume resin bed containing microparticulate silica beads for adsorption and subsequent release of peptides in a reverse phase process. The bed size and flow characteristics permit peptide recovery, for example from 45 to 75% depending on molecular parts per microlitre, from microvolumes of protein-containing materials for such purposes as concentration, desalting and fractionation, but the processing time for the sample remains comparatively slow and the handling of the tips requires care and consequently has an adverse influence on the time factor. Other known micropurification procedures, such as microcolumns with reverse-phase chromatographic materials and pipette tips with reverse-phase beads in glass-fibre structures and throughflow induced by gas pressure, have similar disadvantages with respect to processing time and ease of use.

It is accordingly the principal object of the present invention to provide a method of recovering very small amounts, for example attomole and femtomole amounts, of charged compounds, especially proteinaceous compounds such as peptides, polypeptides and proteins, with improved efficiency by comparison with known methods, particularly with regard to one or more of processing time per unit volume, yield of target substance and concentration of target substance. A further object is integration of the preparation of samples of charged compounds with the process of recovery of compounds from the samples. The method should, in addition, preferably be performable with equipment which is of simple and economic construction and which is relatively uncomplicated to use

Other objects and advantages of the invention will be apparent from the following description.

According to a first aspect of the present invention there is provided a method of isolating a charged compound comprising the steps of providing a prepared sample of the charged compound in solution, placing the prepared sample in contact with a membrane which enables reversible binding thereto of the charged compound in the sample but without binding of the solvent in the sample and which is so limited in effective adsorptive surface area as to preclude irreversible adsorption of at least the substantial part of the bound charged compound, and eluting the charged compound from the membrane.

In the context of the specification, a charged compound is to be understood as any molecule having a net positive or negative charge. It will be appreciated that such compounds may possess a hydrophilic or hydrophobic character or a combination of the two. Charged compounds may include nucleic acids, for example DNA or RNA, and carbohydrates such as aminoglycans. Typically, the charged compound is a proteinaceous compound, that is to say a compound comprising a series of subunits which are bonded together via peptide bonds and typically comprise natural or synthetic amino acids bonded together by peptide bonds forming polymeric compounds, such as peptides, polypeptides and proteins. Such polymeric compounds may be of synthetic or natural origin. It will further be appreciated that the terms ‘proteinaceous compound’ and ‘protein’ may be used interchangeably and consequently may refer to the same thing unless the context of use implies otherwise.

By virtue of the reduced effective adsorptive surface area of the membrane utilised, an irreversible adsorption of the major proportion of bound charged compounds in a lower part of the range of molecular weights thereof is prevented. In particular, the hold-up or solution-retention volume is kept to a smallest size compatible with the chromatographic function of the membrane and the constraints of economic construction, so that loss of target charged compounds, in particular low molecular weight compounds such as peptides of between 500 to 5,000 Daltons, in the membrane body due to, for example, lateral diffusion, is kept to a minimum, thereby allowing recovery of the majority of the bound compounds by a subsequent elution step. The reduced effective area, in conjunction with an appropriately confined receiving volume adjacent to the membrane, also permits in situ preparation of samples at the membrane. The limited effective surface area of the membrane is preferably achieved by a maximum effective external face area of substantially 15 mm², for preference 1 to 3 mm², and a maximum thickness of about 500 microns, preferably 200 to 400 microns, with a pore size of about 0.5 to 5 microns. These dimensions permit use of larger-diameter disc membranes of conventional thickness and nominal pore size of typically 3 to 5 microns, but with an encircling fixing edge zone rendered impermeable and bounding an active adsorptive inner zone with the appropriate face area. The diameter of such an inner zone can thus be about 0.5 to 4 millimetres, preferably 1 to 2 millimetres. The required volume of eluant for effective elution may then be able to be kept down to, for example, 1 to 5 microlitres.

The sample and the eluant are preferably driven through the membrane by applied force, preferably centrifugal force, in which case the membrane can be, for example, part of a spin column and centrifuge tube assembly capable of mounting and spinning in a conventional centrifuge rotor. In one advantageous construction, the membrane is mounted at the base of a spin column or other receptacle with an internal cross-sectional! area reducing in direction towards the membrane at least in a region adjoining the base. Accelerated sample processing can be achieved by applying the sample to the membrane in successively loaded aliquots and intervening action, such as centrifuging, to pass each loaded aliquot through the membrane. Other methods of passaging through the membrane, such as by use of vacuum, wicking or by gas or mechanically applied pressure, are also conceivable.

Processing speed can be further enhanced by passing the sample, for example of a volume of 500 microlitres, through the membrane in aliquots of, for example, up to substantially 200 microlitres, which is far above the amount - typically only 15 to 20 microlitres—possible with conventional procedures employing reverse phase resin bed pipette tips. The preferred aliquot range for membrane loading is substantially 1 to 200 microlitres.

The recovery procedure may in addition be accelerated by elimination of equilibration of the membrane prior to loading with the sample and also, depending on the membrane type, by elimination of washing of the membrane subsequent to passing the sample therethrough and prior to elution of the target compound. Elimination of either or both of these conventional preparatory steps is possible, in appropriate circumstances, due to the small size of the membrane and the expulsion of most of the liquid, thus removing the majority of contaminants, such as buffer salts. The removal of the washing step confers the benefit of avoiding unnecessary loss of target compounds from the membrane matrix.

For elution with acid, the membrane is preferably a basic anion exchanger, which has proved effective for binding compounds over a wide range of molecular weights, particularly peptides with an isoelectric point of 8.0 or less. Elution of bound peptides can be carried out with an acid having a pH reduced relative to that of the sample buffer solution. The membrane can also be a reverse phase membrane, in which case, if elution is by acetonitrile and formic acid, it is desirable to dilute the eluted sample 1:1 to reduce solvent concentration.

The scale of peptide recovery can be increased by passing each of a plurality of prepared samples through a respective one of a corresponding plurality of membranes simultaneously, the membranes being retained by a common support such as a multi-welt plate, and removing the bound peptides by passing eluant through the membranes simultaneously. The force employed to cause passage through the membranes, such as centrifugal force, can thus be exerted to have effect on a number of membranes at the same time.

The method steps may be extended by a concluding step of spectrometric analysis of the eluted charged compound, in which case it is of significant advantage that the compound may be able to be eluted directly onto or into means permitting immediate performance of the analysis.

According to a second aspect of the invention there is provided a method for proteomics analysis comprising the steps of diluting a prepared charged compound digest with a buffer solution, passing the diluted digest through an adsorptive ion exchange or reverse phase membrane so limited in effective adsorptive surface area as to enable reversible binding to the membrane of a subpopulation or subpopulations of charged compounds from the digest at a selected pH or a selection of pH levels, recovering the bound peptides by elution with acid and directly subjecting each peptide eluate to spectrometric analysis.

Such a method is directed to the specific needs of proteomics for a faster and simpler technique of isolation and analysis of large numbers of peptides for such purposes as peptide mass data, amino acid sequencing and post-translational changes including glycosylation changes and phosphorylation. Analysis is preferably carried out by mass spectrometry for characterisation of the peptides present in the eluate, for example matrix-assisted laser desorption ionisation. In that case, depending on the nature of the membrane the eluate may be able to be added to a matrix directly after the elution step, thus without prior washing to eliminate excess salt. Other usable techniques include electrospray (including nanospray) ionisation and also tandem mass spectrometry for peptide and protein identification in, for example, macromolecular complexes

The preparation of proteins for peptide digestion can be carried out with conventional procedures of initially obtaining, purifying (desalting) and separating proteins, especially with use of two-dimensional electrophoresis of a protein-loaded polyacrylamide carrier gel to separate the proteins according to molecular charge and mass.

The membrane characteristics can be as previously described in connection with the first aspect of the invention and corresponding procedures followed as appropriate.

Examples of the present invention will now be particularly described with, respectively, use of an ion exchange membrane and a reverse phase membrane.

### EXAMPLE 1 (ION EXCHANGE MEMBRANE)

A first example of the method of the invention, employing an ion exchange membrane, utilised enolase as test protein, enolase having the merits, apart from a known amino acid sequence, of being commercially available (from Sigma Chemicals Company) in pure form, exhibiting a low cysteine content, being readily capable of digestion with trypsin and allowing production of fragments with a wide range of isoelectric points and masses in the range of 500 to 3500 Da. Specifications of the enolase were in particular:

Enolase=ENO1_YEAST from SWISS-PROT:

ENOLASE 1 (EC 4.2.1.11)(2-PHOSPHOGLYCERATE DEHYDRATASE)(2-PHOSPHO-D-GLYCERATE HYDRO-LYASE). Theoretical isolelectric point (pl): 6.17; Mw (average mass): 46670.92; Mw (monoisotopic mass): 46642.21

Amino Acid Sequence

| | |
|---|---|
| 1 | AVSKVYARSV YDSRGNPTVE VELTTEKGVF RSIVPSGAST GVHEALEMRD |
| 51 | GDKSKWMGKG VLHAVKNVND VIAPAFVKAN IDVSDQKAVD DFLISLDGTA |
| 101 | NKSKLGANAI LGVSLAASRA AAAEKNVPLY KHLADLSKSK TSPYVLPVPF |
| 151 | LNVLNGGSHA GGALALQEFM IAPTGAKTFA EALRIGSEVY HNLKSLTKKR |
| 201 | YGASAGNVGD EGGVAPNIQT AEEALDLIVD AIKAAGHDGK VKIGLDCASS |
| 251 | EFFKDGKYDL DFKNPNSDKS KWLTGPQLAD LYHSLMKRYP IVSIEDPFAE |
| 301 | DDWEAWSHFF KTAGIQIVAD DLTVTNPKRI ATAIEKKAAD ALLLKVNQIG |
| 351 | TLSESIKAAQ DSFAAGWGVM VSHRSGETED TFIADLVVGL RTGQIKTGAP |
| 401 | ARSERLAKLN QLLRIEEELG DNAVFAGENF HHGDKL |

Lyophilised enolase meeting the above specifications was diluted to an approximate concentration of 100 pmol/μl in 10 mM volatile buffer solution of ammonium bicarbonate at ph 7.7. The solution was then subjected to amino acid analysis and finely adjusted by ion to 100 pmol/μl. A peptide digest was then produced by digesting the solution to completion, in particular for 8 hours at 37° C., with modified sequencing grade trypsin (Promega) using a 1:100 ratio of enzyme: substrate. The resultant digest was then diluted concentration of 10 pmol/μl (stock solution) and stored in aliquots of 50 μl at −80° C.

For isolation of peptides from the digest, test buffer solutions of tris (pH 7.3 and 9.0), ammonium bicarbonate (pH 8.0), ethanolamine (pH 10.2) and piperidine (pH 11.4) were made up to a concentration of 20 mM and their pH values adjusted with 1 M hydrochloric acid. Stock solutions of the digest were then diluted with each of the buffer solutions to provide diluted digest solutions of 50 fmol/μl. 100 μl of each pH dilution were loaded into a spin column located in a 1.5 ml microcentrifuge tube, the column containing a strong basic anion exchange membrane with quaternary ammonium functional group covalently bound to a stabilised regenerated cellulose matrix with a nominal 3 to 5 μm pore size. The membrane had the form of a disc of 230 to 320 μm thickness fixed at an impermeable edge zone to leave an effective surface area at each face of approximately 3 mm². If the membrane employed has been preconditioned with salt, any residual salt content should be removed or substantially removed by washing. The tube and column assembly is then placed in a microcentrifuge (‘Biofuge-pico’, Heraeus Instruments, Osterode, Germany) and spun with low relative centrifugal force of less than 300xg for up to 1 minute to drive the respective diluted digest sample through the membrane to produce binding of peptides in the sample in the membrane matrix by anion exchange. The column, without intervening washing of the membrane, was then placed in a fresh centrifuge tube, loaded with 5 μl of 10% formic acid as eluate and spun, together with the tube, in the centrifuge at 300xg for 1 minute and finally at maximum speed for 20 seconds to elute the bound peptides. The resulting eluates were spotted in 0.20 μl volumes onto a thin-layer matrix on a stainless steel target plate for matrix-assisted laser desorption ionisation time-of-flight (MALDI ToF) analysis. The matrix consisted of a 3:1 mixture of a saturated solution of α-cyano4-hydroxycinnamic acid (CCA) in acetone. mixed with a 1:1 mixture of acetone: isopropanol containing 10 mg/ml nitrocellulose.

In addition, for analysis of binding sensitivity, dilutions (0.1, 1, 5 and 10 fmol/μl) of the stock digest were made up in 20 mM ethanolamine buffer with a pH of 10.2. 100 μl of each dilution was spun in a column and tube assembly in the same manner as described above to bind the peptides in the membrane. Elution using 1 μl 10% formic acid gave an increased final volume of 1.5 μl of which 0.1 μl was analysed by MALDI.

Analysis of peptide digests and eluates was carried out on a Reflex III MALDI ToF (Bruker UK Ltd, Coventry) with Scout 384 ion source using a nitrogen laser (λ=337 nm) to desorb/ionise the matrix/analyte material from the sample substrate. Ions generated in this way were allowed to drift for the short delayed extraction time setting before being accelerated by a potential of +25 kV. Spectra were acquired at a microchannel plate detector located after the reflection ion-mirror. Accurate internal calibrations of the spectra were carried out using the masses of peptides known to be produced by a digest of enolase. The calibrated spectra for each sample were searched using the MASCOT search tool (www.matrixscience.com) to identify all the enolase peptides present in each sample.

The results of the MALDI analysis are given in Tables 1 and 2:

TABLE 1
MALDI analysis of peptide solutions at different concentrations, for material that has been concentrated from large
volumes compared with those of the starting material

| Peptides | starting | starting | 500 fmol in 100 μl | 100 fmol in 100 μl | |
|---|---|---|---|---|---|
| known to be | material at | material at | concentrated | concentrated | |
| present | 500 fmol/μl | 5 fmol/μl | to 333 fmol/μl | to 66 fmol/μl | |
| (MW in Da) | (MW in Da) | (MW in Da) | (MW in Da) | (MW in Da) | Sequence |
| 755.47 | | | | | LNQLLR |
| 806.43 | | | 806.43 | 806.43 | TFAEALR |
| 1099.52 | 1099.52 | | 1099.52 | | DGKYDLDFK |
| 1158.6 | 1158.6 | | 1158.6 | | IGSEVYHNLK |
| 1287.7 | | | 1287.7 | | VNQIGTLSESIK |
| 1315.61 | 1315.61 | | 1315.61 | | IGLDCASSEFFK |
| 1411.81 | 1411.81 | 1411.81 | 1411.81 | | LGANAILGSVSLAASR |
| 1415.71 | 1415.71 | | 1415.71 | | GNPTVEVELTTEK |
| 1454.67 | 1454.67 | | 1454.67 | | YDLDFKNPNSDK |
| 1577.79 | 1577.79 | | 1577.79 | | AVDDFLISLDGTANK |
| 1754.94 | 1754.94 | | 1754.94 | | TAGIQIVADDLTVTNPK |
| 1788.84 | 1788.84 | 1788.84 | 1788.84 | | AAQDSFAAGWGVMVSHR |
| 1820.92 | 1820.92 | 1820.92 | 1820.92 | 1820.92 | SGETEDTFIADLWGLR |
| 1839.91 | 1839.91 | 1839.91 | 1839.91 | | SIVPSGASTGVHEALEMR |
| 1871.96 | 1871.96 | 1871.96 | 1871.96 | | WLTGPQLADLYHSLMK |
| 2327.05 | 2327.05 | 2327.05 | 2327.05 | | IEEELGDNAVFAGENFHHGDK |
| 2440.13 | 2440.13 | 2440.13 | 2440.13 | 2440.13 | IEEELGDNAVFAGENFHHGDK |
| 2827.28 | 2827.28 | 2827.28 | | | YPIVSIEDPFAEDDWEAWSHFFK |
| 2983.38 | 2983.38 | 2983.38 | 2983.38 | | RYPIVSIEDPFAEDDWEAWSHFFK |
| 3256.61 | 3256.61 | | 3256.61 | | YGASAGNVGDEGGVAPNIQTAEEALDLIVDAIK |
| 3412.71 | | | | | RYGASAGNVGDEGGVAPNIQTAEEALDLIVDAIK |
| 61% | 52% | 29% | 56% | 10% | Sequence coverage as percentage of total protein |

The data in Table 1 show, in column 1, that 61% of the known protein amino acid sequences were present as peptides in the protein digest, the sequences being given in column 6.

MALDI ToF analysis of the starting material at 500 fmol/μl, i.e. before passing through the membrane, identified 52% of these peptides as being present, as shown in column 2. This is an indication of the variability of mass spectrometric analysis. The sequence coverage obtained shows a correlation with the sample concentration, hence column 3 indicates the lowest dilution at which material could be detected by the MALDI. In that case, the sequence coverage was much lower, i.e. 29%.

Columns 4 and 5 show the peptides recovered after passing the starting material through the membrane and analysing the subsequently obtained eluates. 500 fmol and 100 fmol in 100 μl aliquots, respectively, were loaded and eluted in 1 μl of 10% formic acid. Residual liquid in the membrane yielded a final volume of 1.5 μl, giving effective maximum concentrations of 333 and 66 fmol/μl. 100 nl of each of these eluates was spotted directly onto MALDI target plates. At the 500 fmol concentration there was no loss of sequence coverage compared with the starting material. This result was achieved by concentrating 100 μl of a 5 fmol/μl solution to 1.5 μl in a time interval of less than 3 minutes. The analysis of a 5 fmol/μl sample gave a 29% sequence coverage, indicating a particular utility for proteomics applications where maximum sequence coverage is required.

Finally, column 5 indicates that low amounts of peptides can be concentrated and desalted and are therefore compatible with mass spectrometry.

TABLE 2
Results of MALDI analysis of peptide solutions passed through the membrane at different pH. Peptides are shown in
order of increasing isoelectric point (pI).

| MALDI of | | | | | | | | | |
|---|---|---|---|---|---|---|---|---|---|
| starting | Peptides | | | | | | | Enzyme | |
| material | known to be | Buffer Conditions | | | | | | missed | |
| (Da) | present | pH 7.3 | pH 8.0 | pH 9.0 | pH 10.2 | pH 11.4 | pI | cleavages | Sequence |
| 3256.61 | 3256.61 | 3256.61 | 3256.61 | 3256.61 | 3256.61 | 3256.61 | 3.71 | 0 | YGASAGNVGDEGGVAPNIQTAEEALDLIVDAIK |
| 1820.92 | 1820.92 | 1820.92 | 1820.92 | 1820.92 | 1820.92 | 1820.92 | 3.92 | 0 | SGETEDTFIADLWGLR |
| 1577.79 | 1577.79 | | 1577.79 | 1577.79 | 1577.79 | 1577.79 | 3.93 | 0 | AVDDFLISLDGTANK |
| 2827.28 | 2827.28 | 2827.28 | 2827.28 | 2827.28 | 2827.28 | 2827.28 | 4.01 | 0 | YPIVSIEDPFAEDDWEAWSHFFK |
| | 3412.71 | | 3412.71 | | | 3412.71 | 4.02 | 1 | RYGASAGNVGDEGGVAPNIQTAEEALDLIVDAIK |
| 1754.94 | 1754.94 | | | | 1754.94 | 1754.94 | 4.21 | 0 | TAGIQIVADDLTVTNPK |
| | 1415.71 | 1415.71 | 1415.71 | 1415.71 | 1415.71 | 1415.71 | 4.25 | 0 | GNPTVEVELTTEK |
| 2983.38 | 2983.38 | 2983.38 | 2983.38 | 2983.38 | 2983.38 | 2983.38 | 4.29 | 1 | RYPIVSIEDPFAEDDWEAWSHFFK |
| 2327.05 | 2327.05 | 2327.05 | 2327.05 | 2327.05 | 2327.05 | 2327.05 | 4.35 | 0 | IEEELGDNAVFAGENFHHGDK |
| 2440.13 | 2440.13 | 2440.13 | 2440.13 | 2440.13 | 2440.13 | 2440.13 | 4.35 | 1 | IEEELGDNAVFAGENFHHGDKL |
| | 1315.61 | | 1315.61 | 1315.61 | 1315.61 | 1315.61 | 4.37 | 0 | IGLDCASSEFFK |
| | 1099.52 | | | 1099.52 | 1099.52 | 1099.52 | 4.43 | 1 | DGKYDLDFK |
| 1454.67 | 1454.67 | | | 1454.67 | 1454.67 | 1454.67 | 4.43 | 1 | YDLDFKNPNSDK |
| 1839.91 | 1839.91 | 1839.91 | 1839.91 | 1839.91 | 1839.91 | 1839.91 | 5.38 | 0 | SIVPSGASTGVHEALEMR |
| 806.43 | 806.43 | | | | | | 5.66 | 0 | TFAEALR |
| | 1287.7 | | | | | 1287.7 | 5.97 | 0 | VNQIGTLSESIK |
| 1871.96 | 1871.96 | | 1871.96 | 1871.96 | 1871.96 | 1871.96 | 6.74 | 0 | WLTGPQLADLYHSLMK |
| 1158.6 | 1158.6 | | | | 1158.6 | 1158.6 | 6.75 | 0 | IGSEVYHNLK |
| 1788.84 | 1788.84 | 1788.84 | 1788.84 | 1788.84 | 1788.84 | 1788.84 | 6.79 | 0 | AAQDSFAAGWGVMVSHR |
| 1411.81 | 1411.81 | 1411.81 | 1411.81 | 1411.81 | 1411.81 | 1411.81 | 9.75 | 0 | LGANAILGVSLAASR |
| 755.47 | 755.47 | | | | | | 9.75 | 0 | LNQLLR |

| | | | | | | | |
|---|---|---|---|---|---|---|---|
| 52% | 61% | 34% | 46% | 49% | 56% | 59% | Percentage sequence coverage of protein |

In the case of Table 2, 5 pmol of peptide digest in 100 μl was passed through the membrane to bind peptides, which were then eluted in 5 μl of 10% formic acid in less than 3 minutes. As the pH of the buffer solutions was increased, more peptides of higher pl were bound. Thus, by increasing the loading pH, higher percentage sequence coverages were obtained. This, again, reveals a capability for effectively concentrating and desalting dilute solutions of peptides.

### EXAMPLE 2 (REVERSE PHASE MEMBRANE)

In a second example of the method of the invention, employing a reverse phase membrane, lyophilised enolase as used in Example 1 was processed to a diluted peptide digest in the same manner as described in the preceding example. A range of concentrates of ammonium bicarbonate buffer (20, 50 and 100 mM) were made up and stock solutions of the digest were diluted with each of the buffer solutions to give diluted digest solutions of 50 fmol/μl and 10 fmol/μl. 100 μl of each dilution was loaded into a spin column located in a 1.5 ml microcentrifuge tube and containing a reverse phase membrane having the form of a disc of 230 to 320 μl thickness fixed at an impermeable edge zone to leave an effective surface area at each face of approximately 3 mm². The tube and column assembly were placed in a microcentrifuge (‘Biofuge-pico’, Heraeus Instruments, Osterode, Germany) and spun with low relative centrifugal force of less than 300×g for under 3 minutes to drive the respective diluted digest sample through the membrane and into the collecting chamber of the centrifuge tube to cause retention of peptides in the sample in the membrane. 100 μl of 0.1% formic acid in aqueous solution was then loaded into the column and the column and tube assembly spun for a further period of up to 3 minutes to wash the membrane for removal of buffer salts. The column was then placed in a fresh centrifuge tube and peptides eluted from the membrane by loading 1 to 5 μl of 50% acetonitrile in water containing 0.1% formic acid or trifluoroacetic acid into the column, optionally with 10 mg/ml of MALDI matrix material (α-cyano-4-hydroxycinnamic acid), and spinning the column and tube assembly at up to 300×g for up to 3 minutes and then at maximum speed (approximately 12,000×g) for 20 seconds. In the case of the eluant without added MALDI matrix, 0.25 μl of the resultant eluates were diluted to 1μl with 0.1% formic acid to reduce th acetonitrile concentration and the thus-treated eluates were spotted onto thin layers of matrix material—of the same kind as in Example 1—on MALDI target plates for analysis. In the case of the eluant with added matrix, 0.5 μl of the resultant eluates were spotted directly onto the target plates, where the acetonitrile evaporated to leave a layer of fine crystals with incorporated peptides.

In addition, for analysis of binding sensitivity, dilutions (0.1, 1, 5 and 10 fmol/μl) of the stock digest were made up in 50 mM ammonium bicarbonate buffer with a pH of 7.8. 100 μl of each dilution was spun in a column and tube assembly in the same manner as described above to bind the peptides in the membrane. Elution was carried out using 2 μl 50% acetonitrile containing 0.1% formic acid and 0.25 μl of the eluates diluted to 1 μl with 0.1% formic acid were then applied to matrix material on MALDI target plates for analysis.

The MALDI analysis of peptide digests and eluates was carried out in the same manner as for Example 1.

The use of reverse-phase membranes for sensitive protein recovery may offer the advantages, by comparison with resin bed pipette tips or other silica-packed columns, of reduced equipment production cost, higher flow rates, greater robustness and enhanced processing speed.

### EXAMPLE 3 (ION EXCHANGE MEMBRANE)

In a third example, employing an ion exchange membrane, lyophilised enolase as used in Example 1 was made up to an approximate concentration of 100 pmol/μl in 10mM ammonium bicarbonate, pH 7.7. This solution was then sent for amino acid analysis and the solution finely adjusted by dilution to 100 pmol/μl . 100 pmol of the sample was analysed by sodium dodecyl sulphate polyacrylamide gel electrophoresis (SDS-PAGE). The protein was made to migrate through a slab of polyacrylamide gel by applying an electric field; different proteins migrate in different ways and hence mixtures of proteins can be separated. The separated protein was stained in the gel using 0.1% Coomassie Blue stain in 40% methanol, 10% acetic acid and 50% water and when a band of protein was visible, the gel was de-stained with the same solvent mixture, but with the stain excluded. The gel was then washed extensively with water to remove as many contaminants as possible and especially to reduce the levels of sodium dodecyl sulphate which might interact with the membrane.

The water-washed, blue-stained protein band was excised from the gel and cut into 4×1 mm cubes of gel, so that each piece contained approximately 25 pmol of protein.

A digest was then produced using an ion exchange membrane of the kind employed in Example 1 and with centrifuging of samples by corresponding equipment, in which connection, unless otherwise stated, the centrifuge was set to 300×g and operated for 2 minutes.

For production of the digest, a 1×1 mm cube of gel was placed onto the ion exchange membrane of a 200 μl spin column and 200 μl of 20 mM ammonium bicarbonate, pH 8.0, was added to the gel piece. After it waiting for 15 minutes, the centrifuge was operated to remove the liquid. The steps of adding ammonium bicarbonate, waiting and centrifuging were then repeated three times. 100 μl of 100% acetonitrile was then added to shrink the gel and, after waiting for 10 minutes, the column was centrifuged to remove the acetonitrile. Then 10 μl of a solution containing 100 ng of trypsin in 20 mM ammonium bicarbonate, pH 8.0, were added-to rehydrate the gel piece. After a wait of 15 minutes for complete hydration to occur, 20 μl of 20 mM ammonium bicarbonate, pH 8.0, were added and the column was capped and stored at 37° C. for 3 hours. During this time periodic checks were carried out to ensure that the sample had not run dry and that liquid was not dripping through the membrane. At the expiry of the 3 hours, the sample was diluted—the volume of which should not be greater than 30 μl - with 20 mM ethanolamine pH 10.5 to a final volume of 200 μl. The sample was then centrifuged and 100 μl of 20 mM ethanolamine, pH 10.5, were added. After a wait of 10 minutes the sample was centrifuged. The last three steps of adding 100 μl of 20 mM ethanolamine, waiting and centrifuging were then repeated three times, but in the last centrifuge step a further centrifuging at 13,000×g for 30 seconds was performed. The liquid was then stored as a precaution.

Subsequently, 5 μl of 10% formic acid was added to the membrane to elute the bound peptides and the sample was centrifuged at 300×g for 2 minutes followed by 13,000×g for 30 seconds. The acidic eluant was spotted directly onto a thin layer of matrix on a stainless steel target made from three parts of a saturated solution of a-cyano4-hydroxycinnamic acid (CCA) in acetone mixed with one part of a 1:1 mixture of acetone: isopropanol containing 10 mg/ml nitrocellulose.

MALDI analysis of peptide eluates was carried out in the same manner as for Example 1.

### EXAMPLE 4 (REVERSE PHASE MEMBRANE)

For production of a digest using a reverse phase membrane, the membrane was first wet with 5 μl of 100% acetonitrile followed by 50 μl of 20 mM ammonium bicarbonate, a 1×1 mm cube of gel was placed onto the reverse phase membrane of a 200 μl spin column and 200 μl of 20 mM ammonium bicarbonate, pH 8.0, was added to the gel piece. After waiting for 15 minutes, the column was centrifuged to remove the liquid. The steps of adding 200 μl ammonium bicarbonate, waiting and centrifuging were then repeated three times. 100 μl of 100% acetonitrile was then added to shrink the gel and, after waiting for 10 minutes, the column was centrifuged to remove the acetonitrile. Then 10 μl of a solution containing 100 ng of trypsin in 20 mM ammonium bicarbonate, pH 8.0, were added to rehydrate the gel piece. After a wait of 15 minutes for complete hydration to occur, 20 μl of 20 mM ammonium bicarbonate, pH 8.0, were added and the column was capped and stored at 37° C. for 3 hours. During this time periodic checks were carried out to ensure that the sample had not run dry and to top up with further aliquots of 50 μl of ammonium bicarbonate (dripping of the liquid through the membrane did not matter). At the expiry of the 3 hours, the sample was centrifuged and 100 μl of 20 mM ammonium bicarbonate, pH 8, were added. After a wait of 15 minutes the sample was centrifuged. 10 μl of 10% formic acid were then added and the sample centrifuged. A further 10 μl of 10% formic acid were added and, after a wait of 15 minutes, centrifuging of the sample was repeated. Optionally, the membrane was then washed with 0.1% formic acid in water, for example 2×100 μl, and the sample centrifuged so as to remove any remaining buffer salts.

To elute the bound peptides, 2 to 5 μl of 50% acetonitrile, 0.1% formic acid were added or the same eluant was used, but containing 10 mg/ml of a-cyano-4-hydroxycinnamic acid (CCA). The sample was centrifuged at 300×g for 2 minutes and then at 13,000×g for 30 seconds.

The acidic eluant containing CCA could be applied directly to the MALDI target plate, whereas eluant not containing the matrix CCA could be used for electrospray mass spectrometry.

MALDI analysis of peptide eluates was carried out in the same manner as for Example 1.

### EXAMPLE 5 (REVERSE PHASE MEMBRANE)

In an initial step of preparation of suitable spin columns for this example, a first set of discs each of approximately 3 mm² was cut from a 173 high degree of modification (C18 ligand) reverse-phase membrane sheet—with pore size and thickness similar to those in the preceding. Examples—available from Sartorius GmbH, Germany, and supplied under 70% ethanol, and were fitted into a first set of 200 μl micro-volume spin columns. The rest of the membrane sheet was washed in 2×100 ml volumes of 70% acetonitrile +0.1% formic acid, followed by 2×200 ml volumes of 0.1% formic acid and a second set of discs was cut from this remaining sheet and fitted into a second set of the spin columns. 100 μl of 100% methanol was washed through the membrane disc of each spin column by spinning at 381×g for 1 minute. This was followed by 100 μl of 10 mM ammonium bicarbonate spun through the membrane disc under the same conditions. The columns were stored under 100 μl of 10 mM ammonium bicarbonate until ready for use.

A sample of a characterised digest of Enolase (EC 4.2.1.11) (2-phospho-D-glycerate hydrolase) was prepared at 50 fmol/μl concentration in 10 mM ammonium bicarbonate. 0.25 μl of this starting material was spotted directly onto a thin layer of matrix on a MALDI-ToF target plate. A 1.0 fmol/μl dilution of starting material was made in 10 mM ammonium bicarbonate. After centrifuging the prepared spin columns for 1 min at 381×g to remove the ammonium bicarbonate solution, 100 μl of the 1.0 fmol/μl sample were loaded into each column and spun through the membrane under the normal conditions. The columns were washed with 100 μl of 0.1% formic acid. Bound peptides were eluted with 5 μl of 50% acetonitrile, 0.1% formic acid.

0.25 μl of each eluate was taken, mixed on a MALDI-ToF target plate with 0.25 μl of 5 mg/ml matrix dissolved in 50% acetonitrile, 0.1% formic acid, and allowed to crystallise into dried droplets before analysis. The remaining eluates were then diluted 1:1 with 0.1% formic acid to reduce the acetonitrile concentration to 25%. These were then spotted directly onto a thin layer of matrix that had previously been applied to the target plate. 25% acetonitrile was found to be the highest concentration in the range 10, 20, 25, 30, 40, and 50% that could be used to spot samples onto a thin layer of matrix without the matrix dissolving. The sample spots and starting material spot were analysed by MALDI-ToF.

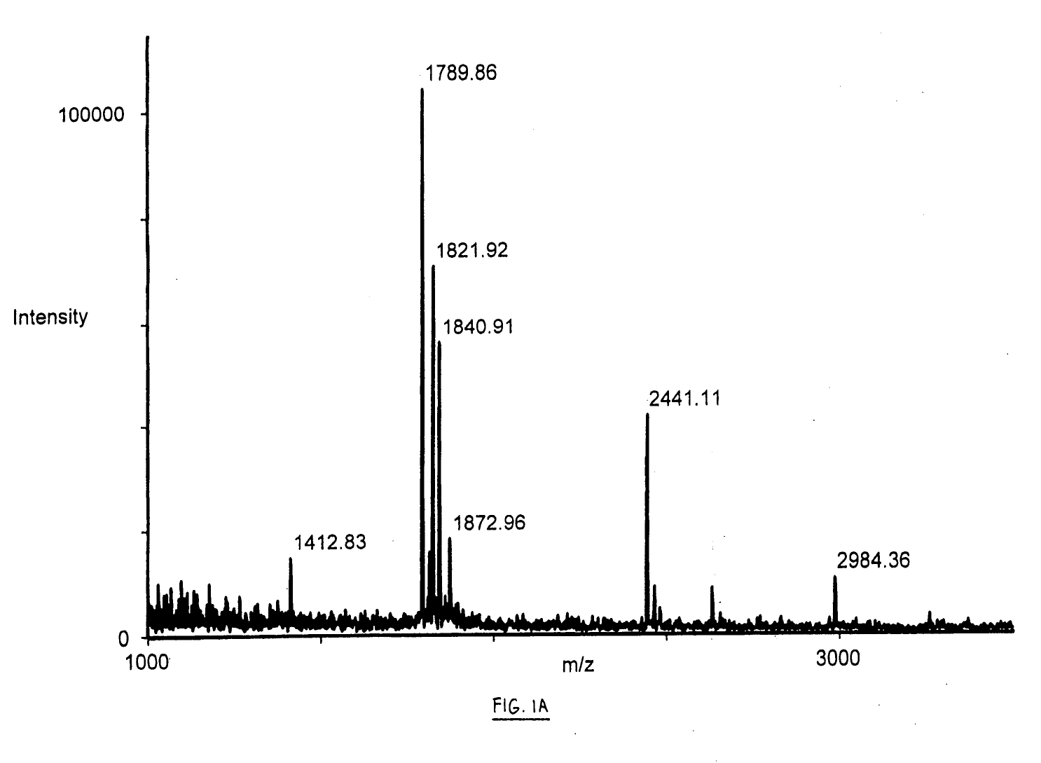

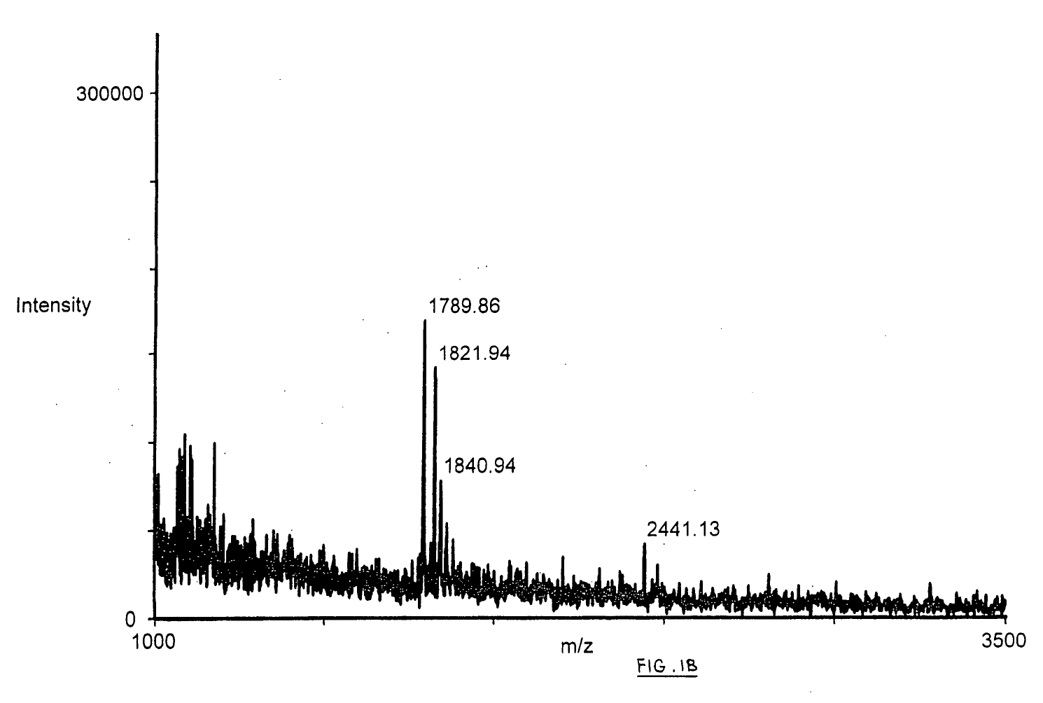

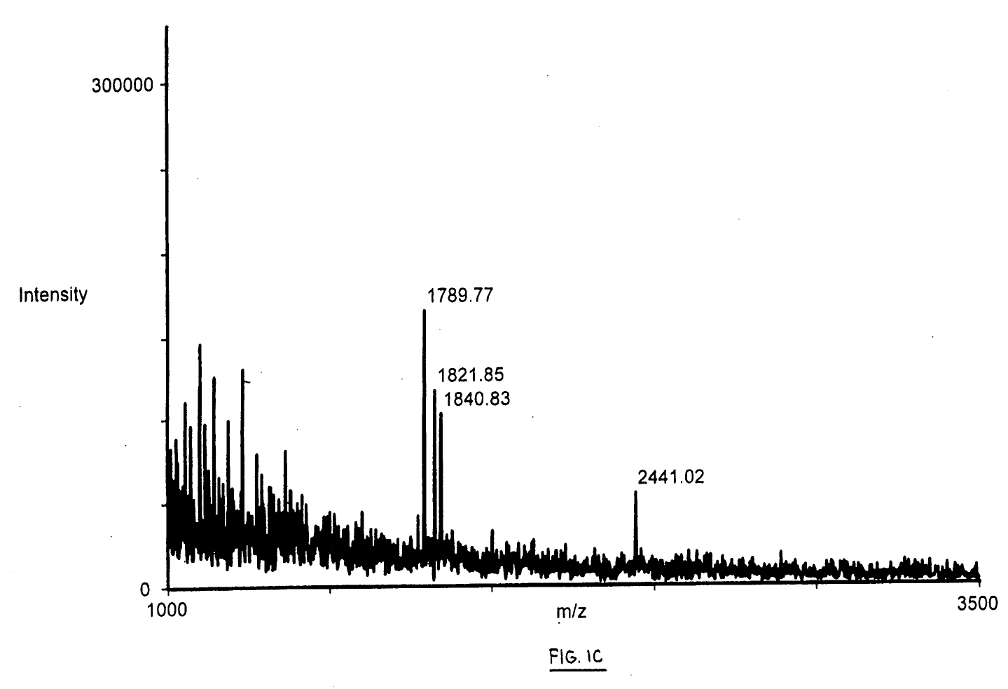

The MALDI-ToF spectra of 100, 50 and 20 fmol samples of the eluates are shown in, respectively, FIGS. 1A, 1B and 1C.

### EXAMPLE 6 (REVERSE PHASE MEMBRANES)

For further characterising the high-modification reverse phase membrane employed in Example 5, 12 spin columns fitted with the membrane were pre-wetted with 100% methanol and washed through with 10 mM ammonium bicarbonate as before. Fresh dilutions of starting material were made at 1.0 fmol/μl, 0.5 fmol/μl, 0.2 fmol/μl and 0.1 fmol/μl in 10 mM ammonium bicarbonate. The prepared spin columns were centrifuged for 1 min at 381×g to remove the ammonium bicarbonate solution. The 12 columns were divided into three sets of four. One column from each set was loaded with 100 μl of 1.0 fmol/μl sample, thus 100 fmol in total, and centrifuged under the normal conditions. The second column from each set was loaded with 100 μl of 0.5 fmol/μl, 50 fmol in total. The third and fourth columns from each set were loaded with 20 and 10 fmol, respectively, from the remaining sample dilutions. Each spin column was then washed with 100 μl of 0.1% formic acid, spun through as before. The columns were then centrifuged again, without refilling, for 20 s at 16,000×g, to remove as much of the aqueous liquid trapped in the membrane pores as possible. 5 μl volumes of 50% acetonitrile, 0.1% formic acid were then used to elute the bound peptides from one set of columns. 2 μl of the same solution were used to elute peptides from the second set of columns. The final set of columns was eluted directly with 2 μl of a solution of 5 mg/ml matrix in 50% acetonitrile, 0.1% formic acid. This last method is directly comparable with the elution method with pipettes, such as ‘Millipore C18 Zip Tips’ (Trade Mark). 0.25 μl of each eluate from the first two sets of columns was taken, mixed on a MALDI-ToF target plate with 0.25 μl of matrix dissolved in 50% acetonitrile and allowed to crystallise into dried droplets. Analysis of these spots is denoted in Table 3 by DD (dried droplet) in the spot method column. The remaining eluates from the first two sets were then diluted 1:1 with 0.1% formic acid to reduce the acetonitrile concentration to 25% and spotted directly onto a thin layer of matrix previously applied to the target plate. Analysis of these spots is denoted in Table 3 by TL (thin layer) in the spot method column. 0.5 μl of eluate from the final set of columns was spotted directly onto the target plate and allowed to crystallise. Analysis of these spots is denoted in Table 3 by DD (pipette equivalent).

The sample spots and starting material spot were analysed by MALDI-ToF using manual data acquisition. On the basis of the spectra recorded, the percentage sequence coverage was calculated from the identified known peptides of enolase. Positive identifications were obtained from the samples as low as 10 fmol loaded in 100 μl, which confirmed the utility of the membrane for analysis of protein digests in the 10 to 50 fmol range, although it is likely that samples of this level would be loaded in 20 μl or less.

TABLE 3

| Spot | Elution | | fmol | Sequence |
|---|---|---|---|---|
| method | Volume | Eluant | loaded | coverage |
| TL | 5 μl | 50% ACN | 100 | 29 |
| TL | 5 μl | 0.1% formic | 50 | 16 |
| TL | 5 μl | diluted 1:1 | 20 | 16 |
| TL | 5 μl | with 0.1% | 10 | 8 |
| | | formic | | |
| TL | 2 μl | 50% ACN | 100 | 15 |
| TL | 2 μl | 0.1% formic | 50 | 20 |
| TL | 2 μl | diluted 1:1 | 20 | 7 |
| TL | 2 μl | with 0.1% | 10 | 0 |
| | | formic | | |
| DD | 5 μl | 50% ACN | 100 | 39 |
| DD | 5 μl | 0.1% formic | 50 | 0 |
| DD | 5 μl | then mixed | 20 | 16 |
| DD | 5 μl | 1:1 with | 10 | 11 |
| | | 5 mg/ml | | |
| | | matrix in | | |
| | | 50% ACN | | |
| | | 0.1% formic | | |
| DD | 2 μl | 50% ACN | 100 | 30 |
| DD | 2 μl | 0.1% formic | 50 | 16 |
| DD | 2 μl | then mixed | 20 | 16 |
| DD | 2 μl | 1:1 with | 10 | 16 |
| | | 5 mg/ml | | |
| | | matrix in | | |
| | | 50% ACN | | |
| | | 0.1% formic | | |
| DD | 2 μl | Elute | 100 | 31 |
| ziptip | | directly with | | |
| equiv. | | 5 mg/ml | | |
| DD | 2 μl | matrix in | 50 | 23 |
| ziptip | | 50% ACN | | |
| equiv. | | 0.1% formic | | |
| DD | 2 μl | | 20 | 16 |
| ziptip | | | | |
| equiv. | | | | |
| DD | 2 μl | | 10 | 16 |
| ziptip | | | | |
| equiv. | | | | |
| TL | 5 μl | Repeats of | 100 | 24 |
| TL | 2 μl | above | 50 | 20 |

1. A method of isolating a charged compound comprising the steps of providing a prepared sample of the charged compound in solution, placing the prepared sample in contact with a membrane which enables reversible binding thereto of the charged compound in the sample but without binding of the solvent in the sample and which is so limited in effective adsorptive surface area as to preclude irreversible adsorption of at least the substantial part of the bound charged compound, and eluting the charged compound from the membrane.

2. A method as claimed in claim 1, wherein the membrane has at each side an effective external face area of at most substantially 15 square millimetres and a thickness of at most substantially 500 microns.

3. A method as claimed in claim 2, wherein the face area is substantially 1 to 3 square millimetres.

4. A method as claimed in any one of the preceding claims, wherein the sample and the eluant are driven through the membrane by force exerted thereon.

5. A method as claimed in claim 4, wherein the force is centrifugal force.

6. A method as claimed in claim 4 or claim 5, wherein the membrane is mounted at the base of a receptacle with an internal cross-sectional area reducing in direction towards the membrane in at least a region adjoining the base.

7. A method as claimed in any one of the preceding claims, wherein the membrane is charged with the sample in successively loaded aliquots and each aliquot is passed through the membrane directly after charging.

8. A method as claimed in any one of the preceding claims, wherein the sample is passed through the membrane in an amount of up to substantially 200 microlitres.

9. A method as claimed in claim 8, wherein the amount is substantially 1 to 200 microlitres.

10. A method as claimed in any one of the preceding claims, wherein the step of passing the sample through the membrane is carried out without prior equilibration of the membrane.

11. A method as claimed in any one of the preceding claims, wherein the step of eluting the bound compounds is carried out without prior washing of the membrane.

12. A method as claimed in any one of the preceding claims, wherein the membrane is a basic anion exchange membrane.

13. A method as claimed in claim 12, wherein the eluant is acid in a buffer solution with a pH value reduced relative to that of the solution containing the sample.

14. A method as claimed in any one of claims 1 to 11, wherein the membrane is a reverse phase membrane.

15. A method as claimed in claim 14, wherein the eluant is acetronitrile and formic acid and the membrane is diluted 1:1 to reduce solvent concentration.

16. A method as claimed in any one of the preceding claims, wherein a plurality of the samples are respectively passed through a corresponding plurality of the membranes simultaneously, the membranes being retained by a common support, and the bound substances are recovered by passing eluant through the membranes simultaneously.

17. A method as claimed in any one of the preceding claims, comprising the step of carrying out spectrometric analysis of the eluted charged compound.

18. A method as claimed in claim 17, wherein the eluted charged compound is eluted directly onto or into means permitting immediate performance of the analysis.

19. A method as claimed in any one of the preceding claims, wherein the charged compound is a proteinaceous compound.

20. A method for proteomics analysis comprising the steps of diluting a prepared charged compound digest with a buffer solution, passing the diluted digest through an adsorptive ion exchange or reverse phase membrane so limited in effective adsorptive surface area as to enable reversible binding to the membrane of a subpopulation or subpopulations of charged compounds from the digest at a selected pH or a selection of pH levels, recovering the bound charged compounds by elution with acid and directly subjecting each charged compound eluate to spectrometric analysis.

21. A method as claimed in claim 20, wherein the analysis is carried out by matrix-assisted laser desorption ionisation.

22. A method as claimed in claim 21, wherein the eluted charged compound is eluted directly onto a matrix for performance of the laser desorption ionisation.

23. A method as claimed in any one of claims 20 to 22, wherein the charged compound is a proteinaceous compound.

24. A method as claimed in any one of claims 23, wherein the characterisation of the proteinaceous compound comprises identifying the amino acid sequences thereof.

25. A method as claimed in any one of claims 20 to 24 wherein the membrane has at each side an effective external face area of at most substantially 15 square millimetres and a thickness of at most substantially 500 microns.

26. A method as claimed in claim 25, wherein the face area is substantially 1 to 3 square millimetres.

27. A method as claimed in any one of claims 20 to 26, wherein the sample and the eluant are driven through the membrane by force exerted thereon.

28. A method as claimed in claim 27, wherein the force is centrifugal force.

29. A method as claimed in any one of claims 20 to 28, wherein the membrane is a basic anion exchange membrane.

30. A method as claimed in any one of claims 20 to 29, wherein the prepared charged compound digest is prepared in situ at the membrane.
